(19) United States
(12) Patent Application Publication
(10) Pub. No.: US 2025/0205249 A1
(43) Pub. Date: Jun. 26, 2025

(54) COMPOSITION FOR USE IN ENHANCING, BOOSTING, RESTORING AND/OR PREVENTING THE DECLINE OF COGNITIVE FUNCTIONS, ADJUVANT COMPOSITION, METHOD FOR USING THE COMPOSITION OR ADJUVANT COMPOSITION, AND KIT HAVING THE COMPOSITION OR ADJUVANT COMPOSITION

(71) Applicant: Instytut Badawczy Innowacyjno Rozwojowy "Biotomed" Sp. Z O.O, Bialystok (PL)

(72) Inventor: Mikolaj Tomulewicz, Bialystok (PL)

(21) Appl. No.: 18/999,164

(22) Filed: Dec. 23, 2024

(30) Foreign Application Priority Data
Dec. 23, 2023 (EP) 23220197.0

(51) Int. Cl. A61K 31/56; A61K 31/51; A61K 31/724; A61K 36/185; A61K 38/48; A61P 25/28

(52) U.S. Cl. CPC A61K 31/56; A61K 31/51; A61K 31/724; A61K 36/185; A61K 38/4893; A61P 25/28

(57) ABSTRACT

A composition includes from 100 mg to 250 mg of betulin and from 580 mg to 1465 mg of γ (gamma)-cyclodextrin for use in at least one of enhancing, boosting, restoring or preventing the decline of cognitive functions. An adjuvant composition, a method of using the composition or adjuvant composition and a kit including the composition or adjuvant composition, are also provided.

### CROSS-REFERENCE TO RELATED APPLICATION

This application claims the priority, under 35 U.S.C. § 119, of European Patent Application EP 23 220 197.0, filed Dec. 23, 2023; the prior application is herewith incorporated by reference in its entirety.

### FIELD AND BACKGROUND OF THE INVENTION

The present invention relates to a composition for use in treatment, enhancing, boosting, restoring and/or preventing the decline of cognitive functions in a subject in need. The invention also relates to an adjuvant composition, a method for using the composition or adjuvant composition as a non-therapeutic dietary supplement for enhancing, boosting and/or restoring memory or learning, and a kit having the composition or adjuvant composition.

Alzheimer's disease (AD) is recognized by the World Health Organization (WHO) as a global priority for public health and poses a serious challenge to the health and social care system. It is a decline of cognitive functions. Despite the significant increase of knowledge about the pathogenesis of AD and how the disease has been conceptualized since Alois Alzheimer described her first case in 1907, there are still no known ways of treating or preventing it. Nowadays and with the current state of knowledge it is an incurable disease. Alzheimer's disease is the cause of dementia, accounting for 50-70% of all cases. Symptoms of Alzheimer's include thinking disorder, speech disorder, and getting lost. Dementia itself as an acquired progressive cognitive impairment sufficient to influence daily activities—is a major cause of addiction, disability and mortality. Current estimates indicate that today there are almost 44 million people with Alzheimer's disease in the world, and another new case is diagnosed every 4 seconds. According to WHO data, by 2030 this number will increase to 65 million, and in 2050 to 115 million. It is estimated that in Poland the population of people over 65 is currently about 14.7%, in 2035 it will increase to 24.5%, and in 2050 it will reach over 30%. According to data from the Central Statistical Office in Poland, there are currently over 300,000 people with Alzheimer's disease, and by 2050 this number will triple and amount to almost 1 million patients. Most cases of Alzheimer's disease appear spontaneously.

Alzheimer's disease is a chronic neurodegenerative disorder characterized by progressive loss of memory and cognitive functions. In people with developing Alzheimer's disease, increasing learning and memory impairment after all leads to the development of a full-blown disease. In a small percentage, problems with language, executive functions, perception (agnosia) or movement (apraxia) are more important than problems with memory.

The cause of most cases of Alzheimer's disease is unknown, with the exception of 1-5% of cases where the disease was genetic. β-amyloid deposition and neurofibrillary tangles, the main neuropathological features of Alzheimer's disease, occur in selectively sensitive areas of the brain, such as the hippocampus and forebrain cortex.

There are several competing hypotheses trying to explain the cause of the disease. Based on these hypotheses, so far few drugs for Alzheimer's disease have been proposed, and moreover their effectiveness is not high enough.

Among the many factors listed as responsible for Alzheimer's disease, the most important seem to be genetic factors, aggregation of β-amyloid and tau protein.

Extracellular deposits of β-amyloid are the primary cause of the disease, which leads to disturbances in interneuronal communication, resulting in the formation of neurotoxins and, consequently, leading to the death of neurons.

β-amyloid, the main component of amyloid plaques, is produced from the amyloid precursor protein (Amyloid Precursor Protein, APP) through a series of protease cleavages, using enzymes called secretases. As a result of this process, N-terminal, shorter fragments are formed, which perform various, important, but not yet recognized functions. The gene encoding the APP protein is mutated by the same type of enzyme that allows HIV infection.

Based on the above hypothesis, it was proposed to introduce some antiretroviral drugs for treatment. Indeed, it was found that the experimental vaccine against the fragment of APP, Aβ1-42 protein removes amyloid plaques, but did not have a significant effect on dementia [Holmes et al., 2008]. The assessment of verubecestat, which inhibits β-secretase 1, responsible for the formation of β-amyloid, was discontinued because no positive clinical effect was observed [Egan et al., 2018].

The search for drugs for the treatment of Alzheimer's disease is still a big problem because there is no specific goal to fight the disease. From many substances, natural compounds as potential drugs have also been studied. Preclinical studies have provided very promising results, matching or surpassing the potency and effect of drugs used in standard clinics.

It is known from Chinese Patent CN106265793 to use an extract of the mangrove plant Acanthus ilicifolius L for the preparation of drugs for the treatment and/or prevention of Alzheimer's disease.

It is known from international Patent Application WO2018/133563, corresponding to U.S. Publication No. 2021/0177923 A1, to use plant extract of Panax and its pharmaceutical composition to make a medicine for chronic heart failure, diabetes or Alzheimer's disease. On the other hand, another International Publication WO2018/096039, corresponding to U.S. Publication No. 2020/0375880 A1, discloses a method for producing an extract of Tagetes L (marigold) enriched with leontopodic acid B and a method for producing a pharmaceutical composition serving in particular as a medicine for the prevention and/or treatment of neurodegenerative disease, in particular Alzheimer's disease.

It is known from European Patent EP 2 712 619, corresponding to U.S. Publication No. 2014/0087008 A1, to use a pharmaceutical composition containing extracts from the species Monsonia sp. for the prevention and/or treatment of dementia with an excellent β-amyloid inhibitory effect, which is known as an Alzheimer's causative agent.

### SUMMARY OF THE INVENTION

It is accordingly an object of the invention to provide an improved composition for use in enhancing, boosting, restoring and/or preventing the decline of cognitive functions, an adjuvant composition, a method for using the composition or adjuvant composition, and a kit having the composition or adjuvant composition, which overcome the hereinafore-mentioned disadvantages of the heretofore-known compositions, methods and kits of this general type and which provide a promising, new and effective adjuvant therapy to use in combination with subeffective doses of anti-AD medications.

With the foregoing and other objects in view there is provided, in accordance with the invention, a composition for use in the treatment, enhancing, boosting, restoring and/or preventing the decline of cognitive functions in a subject in need, the composition comprising:
from 100 mg to 250 mg, especially from 100.00 mg to 250.00 mg of betulin; and
from 580 mg to 1465 mg, especially from 580.00 mg to 1465.00 mg, more especially from 585.94 mg to 1464.85 mg of γ (gamma)-cyclodextrin.

The dependent claims describe particularly useful embodiments of the invention.

It has been found that pentacyclic triterpenes have neuroprotective properties, which can be extremely useful in preventing and treating Alzheimer's disease, in detail when combined with cyclodextrin.

Pentacyclic triterpenes such as betulin can be represented by the following formula:

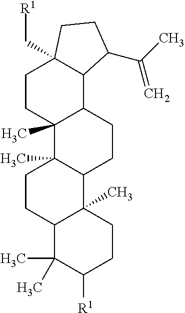

wherein the R¹ substituent is a —OH (betulin) group which can have the cyclodextrin.

According to the invention, a composition for use in the treatment, enhancing, boosting, restoring and/or preventing the decline of cognitive functions in a subject in need, especially a human or an animal, comprises:
from 100 mg to 250 mg, especially from 100.00 mg to 250.00 mg of betulin; and
from 580 mg to 1465 mg, especially from 580.00 mg to 1465.00 mg, more especially from 585.94 mg to 1464.85 mg of γ (gamma)-cyclodextrin.

That means, that the ratio of betulin to γ (gamma)-cyclodextrin may be between 0.16 and 0.18, especially may amount to 0.17, especially to 0.171, 0.172, 0.173, 0.174, 0.175, 0.176, 0.177, 0.178 or 0.179.

Among natural cyclodextrins, γ-cyclodextrin is best soluble in water, while β-cyclodextrin is the least. The solubility of γ-cyclodextrin is 232 mg/mL, at 25° C., while that of β-cyclodextrin is only 18.5 mg/mL, at 25° C.

Orally administered cyclodextrins are non-toxic at low to moderate doses due to lack of absorption from the gastrointestinal tract. Due to nephrotoxicity and lower solubility β-cyclodextrin cannot be administered parenterally.

γ-cyclodextrin is easily digested in the small intestine, while both α- and β-cyclodextrin are digested much more poorly. All parent cyclodextrins are accepted as food additives and “generally recognized as safe” (GRAS). As dietary supplement the total daily oral dose of α-cyclodextrin may reach 6000 mg/day, for β-cyclodextrin 500 mg/day and for γ-cyclodextrin 10,000 mg/day, and for HP-β-cyclodextrin as oral pharmaceutical 8000 mg/day.

In terms of safety of use, to α-cyclodextrin, β-cyclodextrin and its derivative, RM-β-cyclodextrin showed renal toxicity at relatively low doses after parenteral administration and thus are not suitable for medicinal products given intravenously. High doses of >600 mg/kg of γ-cyclodextrin showed only reversible vacuolation in the renal tubular epithelium of rats.

In contrast, the acceptable oral intake of cyclodextrins is defined by the No Observed Adverse Effect Level (NOAEL). This means the highest concentration or amount of a substance at which there are no detectable adverse effects in the exposed population. For β-cyclodextrin, the NOAEL is 10 mg/kg/day, while for γ-cyclodextrin, it is 10 times higher at 100 mg/kg/day.

The composition may comprise from of 111.12 mg to 277.8 mg of an extract, especially purified dry extract from birch bark of Betula pendula, Betula pendula Roth, Betula pubescens, as well as hybrids of these species as equivalent to 0.65 to 1.653 g birch bark, corresponding to 100 mg to 250 mg, especially from 100.00 mg to 250.00 mg betulin.

Advantageously, the composition may comprise further from 0.33 mg to 0.66 mg of thiamine.

Cytotoxicity studies using the XTT assay showed that betulin (1-30 μM) reduced the viability of differentiated SH-SY5Y neuroblastoma cells in a dose-dependent manner and induced a cytotoxic effect (viability below the 70% threshold) above a concentration of 20 μM. In contrast, addition of thiamine increased the survival of SH-SY5Y differentiated neuroblastoma cells over the entire range of concentrations tested (1-30 μM).

Furthermore, a statistically significant protective effect of betulin against differentiated SH-SY5Y cells was observed for concentrations of 1, 2, 5 and 10 μM. The increase in cell viability relative to the EC50 value for the positive control ranged from 12.2 to 15.2%. In contrast, at concentrations of 15 and 20 μM, betulin showed no protective effect and further enhanced the toxic effect of hydrogen peroxide. When thiamine was added, a statistically significant protective effect against differentiated neuroblastoma cells for a toxin concentration equal to EC50 was observed for the entire range of concentrations used, i.e. 1-20 μM. Higher concentrations of the composition, i.e. 20, 15 and 10 μM, improved cell viability by 26.1%-22.5%, while lower concentrations, i.e. 5, 2 and 1, ranged from 19.3 to 16.7%.

Thereby, the amount of the composition or dosage form, administered to the subject in need, especially a human or an animal, may be in the range of 100 to 1000, especially in the range of 100.00 to 1000.00 mg/kg body weight/day, especially from 250 to 500, more especially from 250.00 to 500.00 mg/kg body weight/day.

In detail, 250 mg to 500 mg, especially 250.00 mg to 500.00 mg of the composition may be administered once per day.

The composition may be a pharmaceutical and/or dietary supplement composition comprising an effective amount of the composition.

Further, the decline of cognitive functions may be at least one of the dementia syndrome and the early stages and pre-stages.

Thereby, the dementia syndrome may be selected from light dementia, moderate dementia, Alzheimer dementia, vascular dementia and their mixed forms.

The early stages and pre-stages of dementia may be light cognitive impairments (MCI), cognitive impairments, which are not dementia yet (CIND), subjectively perceived impairments of the memory, subjective impairments of other cognitive components of performance, objectively impaired memory performance, objective impairments of other cognitive components of performance, depressive disorder, anxiety, frightened mood, apathy, lack of motivation, indifference, irritability, excitement, sleeping disorders, and disorders of day and night rhythm.

With the objects of the invention in view, there is also provided an adjuvant composition for use in enhancing, boosting, restoring and/or preventing the decline of cognitive functions in a subject in need, the adjuvant composition comprising a composition as described above, wherein the adjuvant composition is to be administered concurrently, intercurrently, consecutively or concomitantly with at least one of the dementia syndrome and the early stages and pre-stages of dementia or Alzheimer's disease medication, preferably in a subeffective dose.

With the objects of the invention in view, there is furthermore provided a method for using a composition or adjuvant composition according to the invention, as a non-therapeutic dietary supplement for enhancing, boosting and/or restoring memory or learning.

With the objects of the invention in view, there is concomitantly provided a kit-of-parts, comprising the composition or adjuvant composition according to the invention as a non-therapeutic dietary supplement for enhancing, boosting and/or restoring memory or learning.

Other features which are considered as characteristic for the invention are set forth in the appended claims.

Although the invention is illustrated and described herein as embodied in a composition for use in enhancing, boosting, restoring and/or preventing the decline of cognitive functions, an adjuvant composition, a method for using the composition or adjuvant composition, and a kit having the composition or adjuvant composition, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIGS. 1-3 are tables showing test results of studies on the effect of betulin on treatment groups.

### DETAILED DESCRIPTION OF THE INVENTION

### Experimental Section

### Investigation of Betulin Neuroprotective Mechanisms

The study was conducted on Wistar rats weighing 220-250 g. All animals were subjected to two weeks of observation prior to the experiment to exclude infection. Rats were housed in standard cages, 5 individuals in each. The animals stayed in a room with a temperature of 22±2° C., air humidity 60-70% and in natural lighting (12/12 in a day/night cycle). The animals had free access to water and standard laboratory feed ad libitum.

The Alzheimer's disease model was induced by administering aluminum chloride AICl₃ (200 mg/kg body weight, i.g.) to the animals for 120 days. Aluminium is highly neurotoxic, and chronic exposure to AlCl₃ causes a significant increase in the activity of acetylcholinesters, expression of the precursor protein amyloid, TNFα [Rodella et al., 2008; Rather et al., 2018]. Animals were assigned to groups: control group receiving saline (sham operation), group with AlCl₃ without treatment and group with AlCl₃ receiving betulin (100 mg/kg/day, i.g., within the last 50% of the days of the experiment) and betulin in the complex with cyclodextrin (100 mg/kg/day, i.g., in the last 50% of the days of the experiment). Betulin or betulin in a complex with cyclodextrin was administered orally with an intragastric probe in a 2% starch emulsion, reflecting the composition of the finished preparation given in the examples of use.

On the last day of the experiment, the animals were anaesthetized. Brains were quickly collected and then divided into different areas. The entire procedure was carried out in a cold room (+4° C.) on ice, and the samples were frozen in liquid nitrogen and stored in an ultra-low temperature refrigerator for further analysis.

In order to measure rat brain damage caused by aluminum chloride, biochemical determinations were carried out on the collected brain tissue fragments.

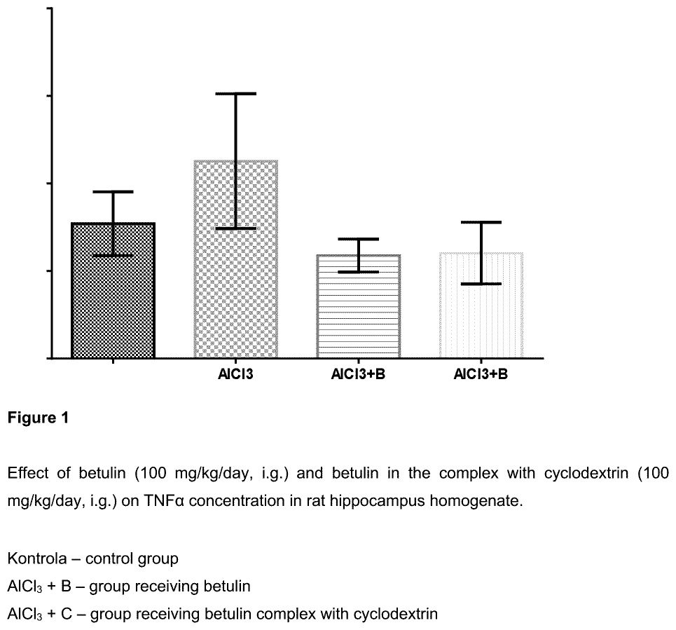

The obtained test results are given in Table 1 and in FIG. 1. Neuroprotective effects were shown in all treatment groups administered betulin (100 mg/kg/day, i.g.) or betulin in the complex with cyclodextrin (100 mg/kg/day, i.g.). Betulin reduced the concentration of TNFα in the hippocampal homogenates of the brains of the animals studied. Much better effects were observed in the group given betulin in the comex with cyclodextrin.

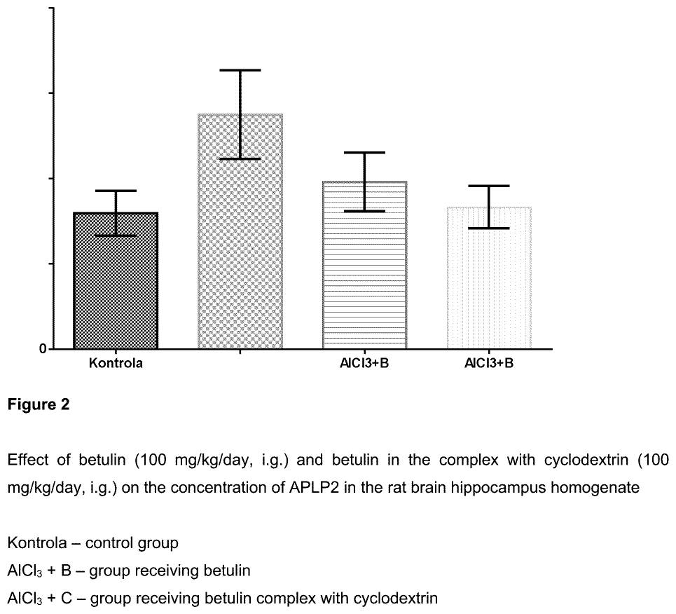

Similarly, Table 2 and FIG. 2 present the results of studies on the effect of betulin on the concentration of the precursor protein APLP2. Neuroprotective effects have been demonstrated in all treatment groups given betulin (100 mg/kg/day, i.g.) or betulin in the complex with cyclodextrin (100 mg/kg/day, i.g.). Betulin reduced the concentration of APLP2 in the hippocampal homogenates of the brains of the animals studied. As in the previous case, much better effects were observed in the group to which betulin was administered in combination with cyclodextrin.

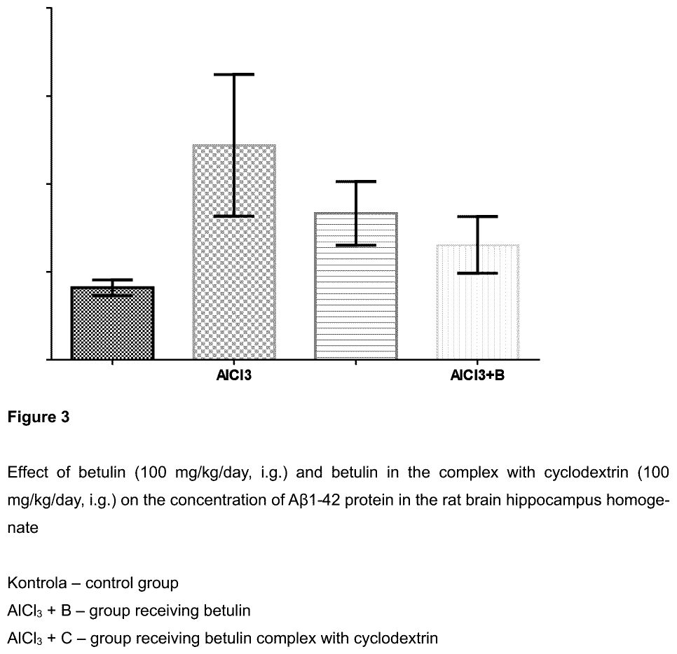

Table 3 and FIG. 3 present the results of studies on the effect of betulin on the concentration of β-amyloid. Neuroprotective effects have been demonstrated in all treatment groups given betulin (100 mg/kg/day, i.g.) or betulin in the complex with cyclodextrin (100 mg/kg/day, i.g.). Betulin reduced the concentration of β-amyloid in the hippocampal homogenates of the brains of the animals studied. Also in this case, much better effects were observed in the group to which betulin was administered in a complex with cyclodextrin.

TABLE 1
Effect of betulin (100 mg/kg/day, i.g.) and betulin
complex with cyclodextrin (100 mg/kg/day, i.g.) on
TNFα concentration in rat hippocampal homogenate.

| Parameter | Control | AlCl₃ | AlCl₃ + B | AlCl₃ + C |
|---|---|---|---|---|
| TNF-α, | 7.70 ± 0.65 | 11.30 ± 1.46 ^{a} | 5.88 ± 0.39 ^{b} | 6.03 ± 0.67 ^{b} |
| pg/mg | | | | |

^{a} p < 0.05 compared to the control group,
^{b} p < 0.05 compared to the AlCl₃ group
AlCl₃ + B—group receiving betulin
AlCl₃ + C—group receiving betulin complex with cyclodextrin

TABLE 2
Effect of betulin (100 mg/kg/day, i.g.) and betulin in the
complex with cyclodextrin (100 mg/kg/day, i.g.) on the concentration
of APLP2 in the rat brain hippocampus homogenate

| Param- | | | | |
|---|---|---|---|---|
| eter | Control | AlCl₃ | AlCl₃ + B | AlCl₃ + C |
| APLP2, | 477.9 ± 24.9 | 824.8 ± 55.1^{a} | 588.1 ± 36.4^{ab} | 499.2 ± 26.3^{b} |
| pg/100 | | | | |
| mg | | | | |

^{a}p < 0.05 compared to the control group,
^{b}p < 0.05 compared to the AlCl₃ group
AlCl₃ + B—group receiving betulin
AlCl₃ + C—group receiving betulin complex with cyclodextrin

TABLE 3
Effect of betulin (100 mg/kg/day, i.g.) and betulin in the
complex with cyclodextrin (100 mg/kg/day, i.g.) on the concentration
of Aβ1-42 protein in rat hippocampal homogenate

| Parameter | Control | AlCl₃ | AlCl₃ + B | AlCl₃ + C |
|---|---|---|---|---|
| Protein Aβ₁₋₄₂, | 40.92 ± 1.56 | 121.90 ± 14.30^{a} | 83.25 ± 6.40^{ab} | 65.34 ± 5.70^{b} |
| pg/100 mg | | | | |

^{a}p < 0.05 compared to the control group,
^{b}p < 0.05 compared to the AlCl₃ group
AlCl₃ + B—group receiving betulin
AlCl₃ + C—group receiving betulin complex with cyclodextrin

Influence of betulin on spatial learning and memory in Morris water maze (MWM) test and a motor coordination in beam walking test in rats with Alzheimer-like neurodegeneration (last testing, 5th day of testing)

The results MWM tests shows that time to reach the platform in the rats as an animal model of AD increased significantly and differed significantly from the results of other animals. The time spent in a target quadrant in rats from the second group was considerably low. The rats from group 2 demonstrated a two-fold increase in both the latency to initiate the task and the total time to cross the beam (p<0.05). Percent of successful trials was only 29% vs 100% in the remaining groups.

TABLE 4
Results of the Morris water maze test and motor coordination
in beam walking test in rats in the experiment

| | Morris water maze test | | Beam walking test | |
|---|---|---|---|---|
| | Parameter | | | |
| | Time to | Time spent | | Percent |
| | reach the | in a target | Time to reach | of |
| | platform, | quadrant. | the shelter, | successful |
| Groups | seconds | seconds | seconds | trials |
| Control | 31 ± 6 | 41 ± 4 | 13.1 ± 2.6 | 100 |
| AlCl₃ | 51 ± 8^{a} | 19 ± 6^{a} | 26.1 ± 5.9^{a} | 29^{a} |
| AlCl₃ + B | 38 ± 7^{b} | 26 ± 8^{ab} | 18.6 ± 3.6^{ab} | 100^{b} |
| AlCl₃ + C | 33 ± 8^{b} | 33 ± 5^{b} | 15.6 ± 3.3^{b} | 100^{b} |

^{a}p < 0.05 compared to the control group,
^{b}p < 0.05 compared to the AlCl₃ group
AlCl₃ + B—group receiving betulin
AlCl₃ + C—group receiving betulin complex with cyclodextrin

Influence of betulin on acetylcholine esterase activity in brain hemispheres and hippocampus in rats with Alzheimer-like neurodegeneration.

One of the treatment method of AD focuses on normalising cholinergic neurotransmission in the brain. The efficacy of acetylcholinesterase inhibitors is explained by acetylcholine's stimulation of muscarinic M1 receptors, which, by activating protein kinase C, increase α-secretase activity and thus prevent the formation of senile plaques. In the AICl₃-induced AD model animals, statistically significantly reduced enzyme activity was observed compared to healthy control animals in both the hippocampus and cerebral hemispheres. From this results, a hypothesis can be drawn that betulin may be an acetylcholinesterase inhibitor, as administration of betulin resulted in a statistically significant increase in acetylcholinesterase activity (p<0.05) in both brain hemispheres and hippocampus to values compared in a control group of healthy animals.

TABLE 5
Acetylcholine esterase activity in brain hemispheres and hippocampus
of rats with AlCl₃-intoxication (200 mg/kg/day of BW, i.g.,
45 days) treated with betulin, μmol/h/mg protein

| Groups | Brain hemispheres | Hippocampus |
|---|---|---|
| Control | 16.95 ± 0.67 | 19.36 ± 0.61 |
| AlCl₃ | 9.76 ± 0.54^{a} | 8.66 ± 0.63^{a} |
| AlCl₃ + B | 16.20 ± 0.55^{b} | 14.22 ± 0.84^{b} |

^{a}p < 0.05 compared to the control group,
^{b}p < 0.05 compared to the AlCl₃ group
AlCl₃ + B—group receiving botulin

Influence of Betulin on Oxidative Stress Parameters in Rats with Alzheimer-Like Neurodegeneration

Oxidative stress is considered one of the causes of AD. Hydroxyl radical inhibits the activity of monoamine oxidases in the brain, which are responsible for the catabolism of neurotransmitters. It also contributes to neuronal loss in brain ischaemia situations in AD. The body has developed specific defence mechanisms to prevent damage caused by ROS. The first line of defence includes the following antioxidant enzymes: superoxide dismutases (SODs), catalase (CAT), glutathione peroxidases (GPxs), glutathione reductase (GR) and glutathione S-transferases (GSTs).

A reduction in GSH, GR and GSTs activity was noted in animals with induced AD. Administration of betulin resulted in a significant increase in the activity of all enzymes. In addition, an increase in GSSG, G6PD and GPO activity was observed in animals with induced AD. Again, administration of betulin reversed this effect and caused a significant decrease in the activity of these enzymes.

TABLE 6
Glutathione content and some antioxidant enzymes activities in brain
hemispheres during Al-intoxication and after treatment with betulin

| | | | | Glutathione | Glutathione- | |
|---|---|---|---|---|---|---|
| | | | | reductase, | S-transferase, | |
| | GSH, | GSSG, | G6PD, nmol | nmol | nmol | GPO, umol |
| | nmol/mg | nmol/mg | NADPH/min/mg | NADPH/min/mg | CDNB/min/mg | GSH/min/mg |
| Groups | protein | protein | protein | protein | protein | protein |
| Control | 28.45 ± 1.25 | 0.11 ± 0.002 | 29.42 ± 1.80 | 11.78 ± 1.27 | 146.90 ± 14.83 | 36.21 ± 3.02 |
| AlCl₃ | 21.16 ± 1.40^{a} | 0.13 ± 0.003^{a} | 34.13 ± 1.42^{a} | 10.61 ± 1.75 | 88.63 ± 9.39^{a} | 65.92 ± 2.41^{a} |
| AlCl₃ + | 26.09 ± 2.19^{b} | 0.112 ± 0.002^{b} | 28.61 ± 1.62^{b} | 11.59 ± 1.39 | 129.90 ± 10.45^{b} | 33.63 ± 2.09^{b} |
| B | | | | | | |

^{a}p < 0.05 compared to the control group,
^{b}p < 0.05 compared to the AlCl₃ group
AlCl₃ + B - group receiving botulin

The test results of Table 6 showing that the invention provides the advantages achieved by such a composition in treatment, enhancing, boosting, restoring and/or preventing the decline in cognitive functions in a subject in need, such like AD.

As part of the work to improve the product, additional studies were carried out: on human neuroblastoma cells of the SH-SY5Y line and a survey of patients taking betulin at a dose of 50-100 mg/day.

### In Vitro

Human neuroblastoma cells of the SH-SY5Y lineage were chosen to perform the experiment. They are a commonly used model for research in human nervous system science. Cells of the SH-SY5Y lineage can be relatively easily differentiated to achieve a neuronal phenotype. Such a condition allows the evaluation of potential neuroprotective properties of applied chemicals, compounds or drugs.

The main advantage of using this cell line is that biological material can be obtained in quantities that allow for much more extensive research work, due to the fact that SH-SY5Y cells have the capacity to proliferate before differentiation. This feature of neuroblastoma cells makes it possible to obtain material to carry out a larger number of repetitions of the planned experiments in order to obtain high statistical significance and thus confidence in the reproducibility, reliability and validity of the results obtained. Ultimately, such a feature will enable researchers to test different administration options of the test chemicals, neurotoxic compounds or drugs, i.e. betulin.

Based on the literature review and research work, hydrogen peroxide H2O2 was chosen as the neurotoxin. Oxidative (and nitrative) stress underlies the majority of CNS disorders, whether genetic, environmental or mixed in origin, and is involved in the pathogenesis of degenerative but often also reversible homeostatic diseases, hence the choice of H₂O₂ as a neurotoxin.

Different routes of administration of the active substance and the neurotoxic substance were tested in cytotoxicity assays on differentiated SH-SY5Y cells (and for the selected variant on human NH neurons), i.e.
i. PREINCUBATION-administration of betulin or reference drug prior to the action of H2O2;
ii. COINCUBATION-simultaneous administration of betulin or reference drug and H2O2;
iii. POSTINCUBATION-delayed administration of betulin or reference drug after H2O2 has been acted upon and neurodegeneration has occurred to see if there is a potential clinically relevant ‘therapeutic window’.

The aim of the pre-incubation variant study was to test whether the active substance (NAC, betulin) has a protective effect on differentiated neuroblastoma cells against a neurotoxic agent (i.e. H₂O₂). Hydrogen peroxide administered to the culture caused a decrease in cell viability by the expected level, i.e. by 50% (for EC50 concentration). In the case of the reference drug, N-acetyl-L-cysteine, 24 h preincubation with the drug resulted in a protective effect when hydrogen peroxide was applied at the EC50 concentration for the entire range of NAC concentrations tested (from 2.5 mM to 0.01 mM). NAC was shown to have a statistically significant (####p<0.001) protective effect on cells (no decrease in viability).

In the case of betulin for the EC50 toxin concentration, a statistically significant protective effect against differentiated SH-SY5Y cells was observed for concentrations of 1, 2, 5 and 10 μM.

The aim of the coincubation variant study was to test whether the active substance (NAC, betulin) exhibits a protective effect against SH-SY5Y differentiated neuroblastoma cells while being exposed to a toxic agent (i.e., H₂O₂).

With the experimental set-up used, after 24 h co-incubation of the drug with hydrogen peroxide at an EC50 concentration, N-acetyl-L-cysteine was shown to have a protective effect at concentrations of 0.01; 0.1 and 1 mM in a dose-dependent manner—the higher the drug concentration, the better the effect on cell viability in culture. The concentrations of NAC tested increased viability by 12, 40 and 54%, respectively, relative to that of cells treated with hydrogen peroxide.

In the case of betulin, a statistically significant improvement in cell viability relative to the positive control was noted for concentrations of 1 and 5 μM, by 27 and 18% respectively.

The aim of the post-incubation variant study was to test whether the active substance (NAC, betulin) exhibits a protective effect against differentiated neuroblastoma cells after the onset of neurodegeneration induced by prior administration of a toxic agent (H₂O₂), to see if there is a potential clinically relevant ‘therapeutic window’.

Two time variants of incubation with hydrogen peroxide in differentiated neuroblastoma culture, i.e. 1 h and 3 h, were used in post-incubation.

It was shown that for both 1 h and 3 h of hydrogen peroxide treatment at EC50 concentration, the subsequent 24 h post-incubation of the culture with the reference drug, N-acetyl-L-cysteine (after partial removal of the toxin) induced a protective effect in a NAC concentration-dependent manner.

In the case of betulin, after treatment with H₂O₂ at EC50 concentration, no increase in viability relative to the positive control was observed for any of the tested concentrations for both time variants.

The analyses also tested whether the neuroprotective effect of the substances tested was related to the inhibition of apoptosis of differentiated SH-SY5Y neuroblastoma cells at an early stage of its induction. A coincubation variant was used, i.e. simultaneous administration of betulin or a reference drug and H₂O₂. H₂O₂ concentrations were EC50 or EC80 (resulting in a 50% and 80% reduction in cell viability in viability assays). Betulin concentrations were 1 μM and 5 μM, while NAC concentrations were 0.1 mM and 1 mM. The coincubation time of the test substances with H2O2 was 24 h.

NAC administered at concentrations of 0.1 mM and 1 mM to cultures of hydrogen peroxide-treated differentiated SH-SY5Y neuroblastoma cells statistically significantly reduced the induction of early apoptosis to 12.2% and 12.0%, respectively, with no clear difference between concentrations. Similarly, administration of betulin to cultures of differentiated hydrogen peroxide-treated neuroblastoma cells reduced the percentage of apotic cells to 2.7% and 2.5% for betulin at concentrations of 1 μM and 5 μM, respectively.

### Clinical Study

Quality of life is an integral part of everyone's being, regardless of age, gender or health status.

The preferred instrument for measuring quality of life in adults is the Euro-Quality of Life Questionnaire (EQ-5D, version EQ5D3L or EQ5D-5L), as its universality of use ensures the greatest comparability of results. The EQ-5D is a general questionnaire used with people aged 12 years and over. It consists of two parts. The first part assesses health status by considering five categories: physical ability, self-care, daily activities, pain and discomfort, depression and depression. For each question, the patient can give one of 5 (in the EQ5D5L version) possible answers: no problems, minor problems moderate problems, major problems inability to perform the given activities. The second part of the EQ5D questionnaire-called the EQ-VAS, contains a visual analogue scale (Visual Analogue Scale, VAS), through the use of which the respondent can assess his/her current state of health on a scale from 0 (worst imaginable health state) to 100 (best imaginable health state). The data obtained with the EQ-5D questionnaire allows the health states of patients suffering from a particular disease entity to be compared with the quality of life assessment of the general population. This allows an objective view of the patient's health status in clinical and economic terms.

Fifty-two participants, 35 women and 17 men, took part in the survey using the EQ-5D-5L questionnaire. The survey was conducted among subjects (n=36) taking 50-100 mg betulin with 0.33 mg to 0.66 mg thiamine and 585.94 mg to 1464.85 mg γ (gamma)-cyclodextrin daily in capsule form, age M=69.5 years, SD=16.8. The control group consisted of subjects (n=16) not taking the test substance, age M=70.1 years, SD=9.76.

To the question Have you experienced memory problems? 50% of the non-responders answered Very Often, while 38.9% of the users answered Very Rarely. This difference was found to be statistically significant, p=0.042. To the question Do you feel that you cannot concentrate on your (daily) activities? 37.5 percent answered often among those not taking the preparation, while among those taking the preparation, 50.0 percent answered Never. This difference was also found to be statistically significant at p=0.075. To the question Does it happen to you that you do not know where you are or how you got here? the same answer was given in both survey groups Never (75.0 5 vs 75.0%). Those taking part in the survey do not need help/assistance from others in their daily functioning (100% of responses Never). Respondents also did not indicate problems with mobility or self-care. Also, when asked about Usual Activities (e.g. work, study, household activities, family activities, leisure activities), both groups of respondents gave a consensus answer of I have no problems performing my usual activities (75.0% vs. 72.2%). In contrast, among the non-users, when asked about pain/discomfort, 43.8% of respondents answered I feel little pain or discomfort. However, to the same question in the group taking the preparation, as many as 61.1% of respondents answered that they did not feel any pain or discomfort. This difference was found to be statistically significant at p=0.002.

The mean VAS score of the non-treated patients was 64.1, while that of the treated patients was 78.3. The difference was statistically significant at p=0.032. Considering the above, it can be concluded that betulin administered in the described composition with thiamine and cyclodextrin at a dose of 50-100 mg/day improves the quality of life of the patients taking it.

Pharmaceutical compositions with neuroprotective effect used in the prevention and treatment of Alzheimer's disease are prepared and administered in various forms. It is obvious to those skilled in the art that unit doses may contain as active ingredient one or more of the compounds described. It is also evident that both dosages and methods of administration will vary depending on the needs of the patient and depending on the particular activity of the active ingredient(s). Determining the dosage and method of administration remains within the skill of each practicing physician.

1. A composition for use in at least one of the treatment, enhancing, boosting, restoring or preventing the decline of cognitive functions in a subject in need, the composition comprising:
from 100 mg to 250 mg of betulin; and
from 580 mg to 1465 mg of γ (gamma)-cyclodextrin.

2. The composition according to claim 1, which further comprises exactly 100.00 mg to 250.00 mg of botulin.

3. The composition according to claim 1, which further comprises exactly from 580.00 mg to 1465.00 mg of γ (gamma)-cyclodextrin.

4. The composition according to claim 1, which further comprises from 585.94 mg to 1464.85 mg of γ (gamma)-cyclodextrin.

5. The composition according to claim 1, which further comprises from 111.12 mg to 277.8 mg of an extract or purified dry extract from birch bark of Betula pendula, Betula pendula Roth, Betula pubescens, as well as hybrids of these species equivalent to 0.65 to 1.653 g birch bark, corresponding to 100 mg to 250 mg of betulin.

6. The composition according to claim 1, which further comprises from 111.12 mg to 277.8 mg of an extract or purified dry extract from birch bark of Betula pendula, Betula pendula Roth, Betula pubescens, as well as hybrids of these species equivalent to 0.65 to 1.653 g birch bark, corresponding to exactly from 100.00 mg to 250.00 mg of betulin.

7. The composition according to claim 1, which further comprises from 0.33 mg to 0.66 mg of thiamine.

8. The composition according to claim 1, wherein an amount of the composition or dosage form, administered to the subject in need, is in a range of 100 to 1000 mg/kg body weight/day.

9. The composition according to claim 1, wherein an amount of the composition or dosage form, administered to the subject in need, is in a range of exactly 100.00 to 1000.00 mg/kg body weight/day.

10. The composition according to claim 1, wherein an amount of the composition or dosage form, administered to the subject in need, is in a range of 250 to 500 mg/kg body weight/day.

11. The composition according to claim 1, wherein an amount of the composition or dosage form, administered to the subject in need, is in a range of from exactly 250.00 to 500.00 mg/kg body weight/day.

12. The composition according to claim 8, wherein 250 mg to 500 mg of the composition is administered once per day.

13. The composition according to claim 8, wherein exactly 250.00 mg to 500.00 mg of the composition is administered once per day.

14. The composition according to claim 1, wherein the composition is at least one of a pharmaceutical or dietary supplement composition including an effective amount of the composition.

15. The composition according to claim 1, wherein the decline of cognitive functions is at least one of the dementia syndrome or early stages or pre-stages of the dementia syndrome.

16. The composition according to claim 15, wherein the dementia syndrome is selected from light dementia, moderate dementia, Alzheimer dementia, vascular dementia and their mixed forms.

17. The composition according to claim 15, wherein the early stages and pre-stages of dementia are light cognitive impairments (MCI), cognitive impairments, which are not yet dementia (CIND), subjectively perceived impairments of memory, subjective impairments of other cognitive components of performance, objectively impaired memory performance, objective impairments of other cognitive components of performance, depressive disorder, anxiety, frightened mood, apathy, lack of motivation, indifference, irritability, excitement, sleeping disorders, and disorders of day and night rhythm.

18. An adjuvant composition for use in enhancing, boosting, restoring and/or preventing the decline of cognitive functions in a subject in need, the adjuvant composition comprising:
a composition according to claim 1;
the adjuvant composition configured to be administered concurrently, intercurrently, consecutively or concomitantly with at least one dementia syndrome or early stage or pre-stage dementia or Alzheimer's disease medication.

19. The adjuvant composition according to claim 18, wherein the adjuvant composition is configured to be administered in a subeffective dose.

20. A method of using a composition according to claim 1, the method comprising using the composition as a non-therapeutic dietary supplement for at least one of enhancing, boosting or restoring memory or learning.

21. A method of using an adjuvant composition according to claim 18, the method comprising using the adjuvant composition as a non-therapeutic dietary supplement for at least one of enhancing, boosting or restoring memory or learning.

22. A kit, comprising the composition according to claim 1 used as a non-therapeutic dietary supplement for at least one of enhancing, boosting or restoring memory or learning.

23. A kit, comprising the adjuvant composition according to claim 18 used as a non-therapeutic dietary supplement for at least one of enhancing, boosting or restoring memory or learning.
